(19) United States
(12) Patent Application Publication
(10) Pub. No.: US 2003/0199404 A1
(43) Pub. Date: Oct. 23, 2003

(54) Hotmelt compositions and related articles

(75) Inventors: Marc Paul Lorenzi, Egham (GB); Aurelie Ledru, Henin-Beaumont (FR); Kristina Emma Inge Vanoosthuyze, Horsell Woking (GB); Mathew Thomas Green, Kingston upon Thomas (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/421,258

(22) Filed: Apr. 23, 2003

Related U.S. Application Data
(60) Provisional application No. 60/374,836, filed on Apr. 23, 2002.

(51) Int. Cl. A61K 7/50

(52) U.S. Cl. 510/119; 510/130

(57) ABSTRACT

The present invention relates to lathering cleansing compositions that comprise: a) a safe and effective amount of one or more mild crystalline surfactants, b) water, and c) a safe and effective amount of one or more polar solvents (other than water) wherein the composition exhibits hotmelt behavior and is substantially free of additional water soluble crystalline structurants. These hotmelt compositions have been found to be useful for cleansing and conditioning skin and hair due to their ability to achieve a sustained lather and to deposit therapeutic benefit agents. The present invention also relates to personal cleansing articles that comprise a water insoluble substrate that is contacted with the above-described hotmelt compositions.

### CROSS REFERENCE TO RELATED APPLICATION

This application claims the benefit of U.S. Provisional Application No. 60/374,836, filed Apr. 23, 2002.

### TECHNICAL FIELD

The present invention relates to lathering compositions suitable for cleansing and therapeutically treating the skin, hair and any other sites in need of such treatment. This lathering composition comprises: a) a safe and effective amount of one or more surfactants that are crystalline at room temperature, b) water, and c) a safe and effective amount of one or more solvents wherein the composition exhibits hotmelt behavior and is substantially free of additional water soluble crystalline structurants. In certain embodiments, the compositions of the present invention may further include therapeutic benefit agents. Alternatively, these benefit agents may be included in an entirely separate composition that is somehow paired with the cleansing compositions of the present invention. For instance, in certain embodiments, the lathering composition for cleansing is included in a substantially dry disposable, nonwoven, personal care article that may further include a therapeutic benefit composition that is separate and apart from the cleansing composition. Such articles may optionally include a therapeutic benefit agent or therapeutic benefit composition containing a therapeutic benefit agent that may be placed in close proximity to the lathering cleansing composition or incorporated directly into the cleansing composition. Consumers use these composition that exhibit hotmelt behavior and/or the related articles by contacting them with water and rubbing them on the area to be cleansed and/or therapeutically treated (e.g., conditioned).

### BACKGROUND OF THE INVENTION

Personal care products, particularly cleansing and conditioning products, have traditionally been marketed in a variety of forms such as bar soaps, creams, lotions, and gels. Typically, these products have attempted to satisfy a number of criteria to be acceptable to consumers. These criteria include cleansing effectiveness, skin feel, mildness to skin, hair, and ocular mucosae, and lather volume. Ideal personal cleansers should gently cleanse the skin or hair, cause little or no irritation, and should not leave the skin or hair with a heavy buildup or overly dry when used frequently.

It is also highly desirable to deliver such cleansing and conditioning benefits from a disposable product. Disposable products are convenient because they obviate the need to carry or store cumbersome bottles, bars, jars, tubes, and other forms of clutter including cleansing products and other products capable of providing therapeutic or aesthetic benefits. Disposable products are also a more sanitary alternative to the use of a sponge, washcloth, or other cleansing implement intended for extensive reuse, because such implements can develop bacterial growth, unpleasant odors, and other undesirable characteristics related to repeated use.

Applicants have surprisingly found that the compositions of the present invention meet these previously unmet needs, especially when applied to a disposable substrate. Applicants have found that the present compositions are not only effective at generating sufficient amounts of lather when used for cleansing but they are also manufactured more efficiently due to their “hotmelt” even in spite of their inclusion of substantial amounts of water. The use of these lathering cleaning compositions that exhibit hotmelt behavior yields the following benefits: 1) speed and ease of manufacture resulting from a one stage liquid tank mixing process; 2) increased processing speed during coating of a disposable substrate with these hotmelt compositions; 3) minimal or no drying time; 4) reduced tackiness (i.e., it and related articles tend to be dry to the touch) and/or reduced strikethrough (i.e., tendency of liquid compositions to penetrate a nonwoven web) due to the solid state of the hotmelt composition at room temperature; 5) improved moldability over softer more liquid-like compositions, e.g., shaped cleansing products can be formed; and 6) highly stable emulsions at room temperature as the solid/semi-solid state of the composition prevents coalescence of emulsified or suspended droplets.

Applicants have also formulated the compositions of the present invention without adding additional water soluble crystalline structurants. Previously known cleansing compositions that exhibit a hotmelt character have typically included additional structurants that aided in the re-crystallization of the compositions on cooling. Conventional structurants usually come in the form of water-soluble crystalline structurants or materials. These materials typically do not have a direct cleansing benefit and/or often exhibit poor surfactancy. These shortcomings often manifest themselves by poor lather and require the addition of synthetic surfactants. Yet, the present compositions have overcome these problems.

It has also been found that the hotmelt behavior of the present compositions allows them to last longer when used during bathing versus a typical liquid cleansing composition. This extended life is primarily due to the semi-solid nature that the hotmelt character lends to the composition thereby slowing its dissolution.

Applicants have also found that although in preferred embodiments the present invention is suitable for personal care applications, the composition may also be useful in a variety of other industries such as the automotive care, marine vehicle care, household care, animal care, etc. where surfaces or areas are in need of cleansing and/or application of a benefit agent, e.g., wax, conditioner, UV protectant, etc.

In preferred embodiments of the present invention, the composition is suitable for personal care applications and is useful for cleansing and optionally conditioning the skin, hair, and similar surfaces in need of such treatment. Consumers can use this composition by disposing them onto a substrate, wetting the composition with water and rubbing on the area to be treated.

### SUMMARY OF THE INVENTION

The present invention relates to a lathering, cleansing composition comprising:

a) a safe and effective amount of one or more mild crystalline surfactants;

b) water, and

c) a safe and effective amount of an polar solvent for the surfactant other than water, and

wherein the composition is exhibits hotmelt behavior and is substantially free of additional water-soluble crystalline structurants besides crystalline surfactants.

In another embodiment, the composition of the present invention further comprises therapeutic benefit agents. These benefit agents may be directly emulsified or incorporated into the cleansing compositions to effectuate a two-in-one system (i.e., a cleansing and conditioning system).

In another embodiment, the present invention further relates to a substantially dry, disposable personal care article suitable for cleansing and conditioning wherein the article comprises the composition of the present invention.

The present invention also relates to a method of cleansing and additionally conditioning the skin and hair.

### DETAILED DESCRIPTION OF THE INVENTION

All percentages and ratios used herein, unless otherwise indicated, are by weight of the total composition and all measurements made are at 25° C., unless otherwise designated. The invention hereof can comprise, consist of, or consist essentially of, the essential)as well as optional ingredients and components described therein.

As used herein “hotmelt” describes a material that undergoes a transition from a solid or semi-solid state at a lower temperature, to a liquid or semi-liquid state at a higher temperature (the extent to which can be physically measured Theologically by a skilled artisan) and then returns to the initial state when cooled. Preferably a composition that exhibits “hotmelt” behavior exhibits a log [(η@25° C.)/(η@95° C.)] greater than about 0.45. In another embodiment, the cleansing component additionally exhibits complex viscosity measured under an oscillation stress of 1 Pa of greater than about 100 Pas. at 25° C.

In the description of the invention various embodiments and/or individual features are disclosed. As will be apparent for the skilled practitioner all combinations of such embodiments and features are possible and can result in preferred executions of the invention.

All documents referred to herein, including patents, patent applications, and printed publications, are hereby incorporated by reference in their entirety in this disclosure.

The compositions of the present invention, including the essential and optional components thereof, are described in detail hereinafter.

Crystalline Surfactant

The present invention comprises a safe and effective amount of one or more mild crystalline surfactants. As used herein “mild crystalline surfactants” refer to those surfactants which are both crystalline and solid or semi-solid at room temperature even when aqueous (i.e., in the presence of water). Many surfactants used in personal cleansing applications do not usually exhibit such behavior in the presence of water. For instance, sodium laureth-2 sulfate is a thin liquid when 25% active in water (a spherical micelle configuration), and becomes a considerably thicker gel with increased concentration as different phases form (from rod micelles to liquid crystalline phases) until it forms a thick pourable gel at 70% active in water. Although this gel is a liquid crystalline phase that thins with increased temperature, it never exhibits a true solid or semi-solid nature in the presence of water and hence would not be an appropriate mild crystalline surfactant for the compositions of the present invention. Applicants have found that certain surfactants have the unique ability to combine both hotmelt properties and be a crystalline solid or semi-solid in nature at room temperature (25° C.) when aqueous. Such surfactants often exhibit a high Kraft point and are opaque crystalline solids or semi-solids at 25° C. and aqueous. Surprisingly, Applicants have also found that adding dilute co-surfactants that are not solid at 25° C. to the said crystalline surfactant does not impair the hotmelt behavior or character of the final composition and its ability to be a solid or semi-solid at 25° C. A typical example of a desirable crystalline surfactant is sodium cocoglyceryl ether sulfonate, which is a semi-solid and crystalline at 25° C. even in the presence of at least 40% water. In particular, this mild crystalline surfactant contributes to the remeltable nature of the composition. Therefore, the hotmelt composition easily melts and resolidifies upon cooling.

The mild crystalline surfactant of the present invention is preferably a lathering surfactant. It is, however, possible that the co-surfactants that may be optionally added to the compositions of the present invention may also be lathering. As used herein, “lathering surfactant” means a surfactant, which when combined with water and mechanically agitated generates a foam or lather. Such surfactants are preferred since increased lather is important to consumers as an indication of cleansing effectiveness. In certain embodiments, the surfactants or combinations of surfactants are mild. As used herein, “mild” means that the surfactants of the present invention demonstrate skin mildness comparable to a mild alkyl glyceryl ether sulfonate (AGS) surfactant based synthetic bar, i.e., synbar. Methods for measuring mildness, or inversely the irritancy, of surfactant containing articles, are based on a skin barrier destruction test. In this test, the milder the surfactant, the lesser the skin barrier is destroyed. Skin barrier destruction is measured by the relative amount of radio-labeled (tritium labeled) water (3H-H₂O) that passes from the test solution through the skin epidermis into the physiological buffer contained in the diffusate chamber. This test is described by T. J. Franz in the J. Invest. Dermatol., 1975, 64, pp. 190-195; and in U.S. Pat. No. 4,673,525, to Small et al., issued Jun. 16, 1987. Other testing methodologies for determining surfactant mildness well known to one skilled in the art can also be used.

Suitable crystalline surfactants for use herein are selected from the group consisting of alkyl glyceryl sulfonates, alkyl glucosides, glucose amides, alkyl lactylates, dialkyl sulfosuccinates, acyl glutmates, alkyl isethionates and combinations thereof.

More preferred surfactants are selected from the group consisting of n-methyl glucose amides (commercially available as Medialan GAL from Clariant), alkyl isethionates (e.g. Hostapon SCI 85 from Clariant), dialkyl sulfosuccinates (e.g. Rewopol SBF 12P from Goldschmidt) alkyl glyceryl ether sulfonates (e.g., sodium cocoglyceryl ether sulfonate from Procter & Gamble), and combinations thereof.

Preferably, the compositions of the present invention comprise from about 15% to about 45%, by weight of the composition, of a crystalline surfactant, more preferably, from about 20% to about 40%, and most preferably, from about 25% to about 35%.

Water

The compositions additionally require the inclusion of water. Surprisingly, Applicants have been able to formulate the compositions of the present invention as aqueous hotmelt systems, which is atypical. The inclusion of water into hotmelt compositions typically precludes a composition from exhibiting hotmelt characteristics. Despite the inclusion of water in the compositions of the present invention, Applicants have developed a true hotmelt system that re-solidifies when cooled to 25° C. within a relatively short period of time without the need for any mechanical drying means.

Unlike most milled bar soaps, the compositions of the present invention comprise at least about 15%, by weight of the composition, of water, preferably at least about 25%, and most preferably at least about 30%.

Polar Solvents

The lathering, cleansing compositons of the present invention also comprise a safe and effective amount of one or more polar solvents. As used herein relative to the solvent “polar” means that the solvent is highly water soluble but is not primarily composed of water. Suitable solvents into which the crystalline surfactants and any additional water-soluble components dissolve during mixing include, but are not restricted to liquid glycols like ethylene glycol, propylene glycol, butylene glycol; glycerol; polyethylene glycols (PEG'shaving a molecular weight of about 1000 or less) like Carbowax PEG 200, C2-C10 ethoxylated alcohols, and C1-C10 propoxylated alcohols.

Preferably, the compositions of the present invention comprise from about 1% to about 25%, by weight of the composition, of the polar solvent, more preferably, from about 5% to about 20%, and most preferably, from about 10% to about 15%.

Hotmelt Behavior

It should be noted that it is imperative to the compositions of the present invention that they exhibit hotmelt behavior. The term “hotmelt” is described above. It is preferred that relative to the hotmelt behavior that the composition exhibit a log [(η@25° C.)/(η@80° C.)] greater than about 0.45, preferably greater than about 1.5, even more preferably greater than about 3, still more preferably greater than about 4, and most preferably greater than about 5. In the most preferred embodiments of the present invention the composition exhibits a log[(η@25° C.)/(η@60° C.)] greater than about 0.45, preferably greater than about 1.5, even more preferably greater than about 3, still even more preferably greater than about 4, and most preferably greater than about 5. Over the latter temperature range, it has been discovered that many of the chemicals that might also be included in the cleansing composition also tend to exhibit improved stability in the composition and are less prone to chemical breakdown due to the lower temperatures.

The individual viscosities, η, are determined at the respective temperatures using a viscometer (e.g., commercially available from TA Instruments, model number AR1000). The measurements are made with a parallel plate having a diameter of 40mm and a gap of 1.25 mm. First, the cleansing composition sample is loaded onto the rheometer plate with the plate set to a temperature sufficiently high to melt crystalline surfactants in the composition, preferably to the higher temperature of interest +20° C. e.g. if interested in ratio between viscosities at 60° C. and 25° C., the plate would be set to 80° C. (as is obvious to one skilled in the art, the high end temperature should not exceed 100° C. so as not to boil the aqueous composition, preferably a higher temperature of interest should be used which does not cause obvious decomposition or evaporation in the sample. The measurement should be set to commence after 180 seconds of equilibration. The viscosity is measured using a rotation at a constant stress of 50 Pa over a temperature sweep from the higher temperature of interest +20° C. down to 20° C. with the temperature changing at a rate of 1° C. every 10 seconds. The viscosities of interest can be read from the resultant temperature viscosity curve. As would be apparent to a skilled artisan, if the viscometer stops during the cooling experiment due to the sample becoming too viscous, due the angular velocity being too low, it means that the sample has become too viscous. The highest viscosity recorded before the experiment stopped should be used for the lower temperature viscosity value in the ratio or the experiment should be repeated using a higher stress, smaller diameter plate or rheometer with higher torque capability.

In support of the “hotmelt” characterization of the compositions, they preferably exhibit a complex viscosity measured under an oscillation stress of 1 Pa of greater than about 100 Pa.s., more preferably, greater than about 500 Pa.s., even more preferably, greater than about, 1000 Pa.s., and most preferably, greater than about 2000 Pa.s., all at 25° C. This complex viscosity is measured utilizing a oscillation method for a flow curve from 0.01/s to 100/s.

The method used to determine the complex viscosity at 25° C. is as follows: The cleansing composition sample should first be prepared by cooling it at a rate equivalent to that used when dispensing into the final product form, e.g., a table or coating the composition onto a substrate during production of finished articles, to ensure that the sample is in a representative physical form. The complex viscosity of the sample is measured using a rheometer such as an AR1000 from TA instruments. The cleansing composition is loaded onto the rheometer plate with the plate set to 25° C. The measurement is taken using a parallel plate of 40 mm diameter and a gap of 400 μm. The complex viscosity is measured under a constant 1 Pa stress oscillation at 2.5 Hz and the reading taken after 30 seconds equilibration. As is obvious to a someone experienced in the art, if the sample is too viscous to give a reading under these conditions, increasing stress should be used until a reading can be made and as long as the stress remains in the linear viscoelastic region.

Both the complex viscosity and individual viscosity methods of characterization can be measured rheologically by a skilled artisan, using instruments such as a TA instruments AR1000 Rheometer with a 4 cm flat parallel plate and a 1000 μm gap with a rotational stress of 50 Pa.

As used herein, “substantially free of additional water-soluble crystalline structurants besides crystalline surfactants” means that the composition of the present invention comprises less than about 5%, by weight of the composition, of water-soluble crystalline structurants besides those crystalline surfactants that are described herein, preferably, less than about 2%, and most preferably less than about 1%. Such structurants known to those skilled in the art of bar & pour molded soap making include fatty acids (most commonly sodium stearate, tallowate & palmitate) fatty alcohols, high molecular weight polyethylene glycols, (MW=1,500 and above) and PEG derivatives (e.g., PEG-10 stearate), ethoxylated and propoxylated alcohols, (C12 and above).

Product Forms

The compositions of the present invention may be presented to a consumer in a variety of product forms. Those product forms include, but are not limited to, single dose tablets, single dose capsules, substantially dry personal cleansing substrate articles, cleansing composition-loaded polymeric mesh sponges, etc. It is envisioned that the tablet and capsule forms could be used via submersion into water during the bathing or showering experience and could be paired with a washing implement. The substrate articles and mesh sponges would inherently include a cleansing implement and could therefore simply be contacted with water for use during a cleansing experience. For those substrate or sponge based product forms of the present invention, suitable substrates are disclosed hereafter.

Water Insoluble Substrate

The articles of the present invention comprise the above described lathering, cleansing compositions that are contacted with a water insoluble substrate. In certain embodiments, the water insoluble substrate includes one or more nonwoven layers. The nonwoven layer is suitable for contact with the skin in which case it is preferred that the layer is soft to the skin.

Preferably, the nonwoven layer is a batting layer. More preferably, the batting layer comprises synthetic materials. Suitable synthetic materials include, but are not limited to, acetate fibers, acrylic fibers, cellulose ester fibers, modacrylic fibers, polyamide fibers, polyester fibers, polyolefin fibers, polyvinyl alcohol fibers, rayon fibers, polyethylene foam, polyurethane foam, and combinations thereof. Preferred synthetic materials, particularly fibers, may be selected from the group consisting of nylon fibers, rayon fibers, polyolefin fibers, polyester fibers, and combinations thereof. Preferred polyolefin fibers are fibers selected from the group consisting of polyethylene, polypropylene, polybutylene, polypentene, and combinations and copolymers thereof. Preferred polyester fibers are fibers selected from the group consisting of polyethylene terephthalate, polybutylene terephthalate, polycyclohexylenedimethylene terephthalate, and combinations and copolymers thereof. Suitable synthetic materials may include solid single component (i.e., chemically homogeneous) fibers, multiconstituent fibers (i.e., more than one type of material making up each fiber), and multicomponent fibers (i.e., synthetic fibers which comprise two or more distinct filament types which are somehow intertwined to produce a larger fiber), and combinations thereof. Preferred fibers include bicomponent fibers, multiconstituent fibers, and combinations thereof. Such bicomponent fibers may have a core-sheath configuration or a side-by-side configuration. In either instance, the batting layer may comprise either a combination of fibers comprising the above-listed materials or fibers which themselves comprise a combination of the above-listed materials.

Additionally, materials suitable for the nonwoven layer are selected from the group consisting of cellulosic nonwovens, non-lofty nonwovens, sponges (i.e., both natural and synthetic), formed films, and combinations thereof. Preferably, the nonwoven layer comprises materials selected from the group consisting of cellulosic nonwovens, non-lofty nonwovens, formed films, foams, sponges, reticulated foams, vacuum-formed laminates, scrims, polymeric nets, and combinations thereof. More preferably, the nonwoven layer comprises materials selected from the group consisting of cellulosic nonwovens, non-lofty nonwovens, formed films, and combinations thereof. As used herein, “nonwoven” means that the layer does not comprise fibers that are woven into a fabric and the layer need not comprise fibers at all, e.g., formed films, sponges, foams, scrims, etc. When the layer comprises fiber, the fibers can either be random (i.e., randomly aligned) or they can be carded (i.e., combed to be oriented in primarily one direction). Furthermore, the nonwoven layer can be a composite material composed of a combination of additional layers, i.e., plies, of random and carded fibers.

In a preferred embodiment, the nonwoven layer is apertured. The apertures in the nonwoven layer of the water insoluble substrate will generally range in average diameter between about 0.5 mm and 5 mm. More preferably, the apertures will range in size between about 1 mm to 4 mm in average diameter. Preferably, no more than about 10% of the apertures in the nonwoven layer of the substrate will fall outside these size ranges. More preferably, no more than about 5% of the apertures in the nonwoven layer will fall outside these size ranges.

The nonwoven layer may comprise a variety of both natural and synthetic fibers or materials. Nonlimiting examples of natural materials useful in the present invention include, but are not limited to, silk fibers, keratin fibers and cellulosic fibers.

Natural material nonwovens useful in the present invention may be obtained from a wide variety of commercial sources. Nonlimiting examples of suitable commercially available paper layers useful herein include Airtex®, an embossed airlaid cellulosic layer having a base weight of about 71 gsy, available from James River, Green Bay, Wis.; and Walkisoft®, an embossed airlaid cellulosic having a base weight of about 75 gsy, available from Walkisoft U.S.A., Mount Holly, N.C.

Additional suitable nonwoven materials include, but are not limited to, those disclosed in U.S. Pat. Nos. 4,447,294, issued to Osborn on May 8, 1984; 4,603,176 issued to Bjorkquist on Jul. 29, 1986; 4,981,557 issued to Bjorkquist on Jan. 1, 1991; 5,085,736 issued to Bjorkquist on Feb. 4, 1992; 5,138,002 issued to Bjorkquist on Aug. 8, 1992; 5,262,007 issued to Phan et al. on Nov. 16, 1993; 5,264,082, issued to Phan et al. on Nov. 23, 1993; 4,637,859 issued to Trokhan on Jan. 20, 1987; 4,529,480 issued to Trokhan on Jul. 16, 1985; 4,687,153 issued to McNeil on Aug. 18, 1987; 5,223,096 issued to Phan et al. on Jun. 29, 1993 and 5,679,222, issued to Rasch et al. on Oct. 21, 1997.

The nonwoven layer may also be a polymeric mesh sponge as described in U.S. Pat. No. 5,977,039 issued to Gordon et al. on Nov. 2, 1999. Such polymeric mesh sponges comprise a plurality of plies of an extruded tubular netting mesh prepared from nylon or a strong flexible polymer, such as addition polymers of olefin monomers and polyamides of polycarboxylic acids.

The nonwoven layer may also comprise formed films and composite materials, i.e., multiple materials containing formed films. Suitable formed films and formed film-containing composite materials useful in the nonwoven layer of the present invention include, but are not limited to, those disclosed in U.S. Pat. No. 4,342,314 issued to Radel et al. on Aug. 3, 1982, U.S. Pat. No. 4,629,643, issued to Curro et al. on Dec. 16, 1986, U.S. Pat. Nos. 6,025,049 and 6,180,052, both issued to Gordon et al. on Feb. 15, 2000 and Jan. 30, 2001, respectively.

Additionally, the nonwoven layer and any additional layers are preferably bonded to one another in order to maintain the integrity of the article. This bonding may consist of spot bonding (e.g., hot point bonding), continuous joining (e.g., laminated, etc.) in a discontinuous pattern, or by bonding at the external edges (or periphery) of the layers and/or at discrete loci or combinations thereof. When spot bonding is used in the present articles, it is preferred that the spot bonds are separated by a distance of not less than about 1 cm. In any instance, however, the bonding may be arranged such that geometric shapes and patterns, e.g. diamonds, circles, squares, etc., are created on the exterior surfaces of the layers and the resulting article.

It is also envisioned in the articles of the present invention that the nonwoven layer and any additional layers may be surface modified to form single composite layer having 2 sides with different textures. Thus, in effect, the water insoluble substrate can be construed as comprising a single composite layer with dual textured sides or surfaces.

In any event, it is preferred that the bonded area present between the nonwoven layer and any additional layers be not greater than about 50% of the total surface area of the layers, preferably not greater than about 15%, more preferably not greater than about 10%, and most preferably not greater than about 8%.

The nonwoven layers that may be included in the articles of the present invention comprise at least two surfaces, namely an interior surface and an exterior surface, each of which may have the same or different texture and abrasiveness. Preferably, the articles of the present invention comprise substrates and therefore layers which are soft to the skin. Differing texture substrates can result, however, from the use of different combinations of materials or from the use of different manufacturing processes or a combination thereof. For instance, a dual textured water insoluble substrate can be made to provide a personal care article with the advantage of having a more abrasive side for exfoliation and a softer, absorbent side for gentle cleansing and/or therapeutic treatment. In addition, the separate layers of the substrate can be manufactured to have different colors, thereby helping the user to further distinguish the surfaces.

Moisture Retention Methodology

As described above, the articles of the present invention are considered to be “substantially dry”. As used herein, “substantially dry” means that the articles of the present invention exhibit a Moisture Retention of less than about 0.95 gms, preferably less than about 0.75 gms, even more preferably, less than about 0.5 gms, even more preferably less than about 0.25 gms, even still more preferably less than about 0.15 gms, and most preferably, less than about 0.1 gms. The Moisture Retention is indicative of the dry feel that users perceive upon touching the articles of the present invention as opposed to the feel of “wet” wipes.

In order to determine the Moisture Retention of the present articles and other disposable substrate-based products, the following equipment and materials are needed.

| | |
|---|---|
| Bounty White Paper Towel | Procter & Gamble SKU 37000 63037 |
| | Basis Weight = 42.14 gsm |
| Balance | Accurate to 0.0 g |
| Lexan | 0.5” thickness |
| | large enough to cover samples |
| | completely and weighs 1000 g |
| Weight | A 2000 g weight or combination to |
| | equal 2000 g |

Next, weigh two paper towels separately and record each weight. Place one paper towel on flat surface (e.g., lab bench). Place the sample article on top of that towel. Place the other paper towel on top of sample article. Next, place the Lexan and then the 2000 g weight(s) on top of the sandwiched sample article. Wait 1 minute. After the minute, remove weight(s) and Lexan. Weigh the top and bottom paper towel and record the weight.

Calculate the Moisture Retention by subtracting the initial paper towel weight from the final weight (after 1 minute) for both the top and bottom paper towels. Add the weight differences obtained for the top and bottom paper towels. Assuming multiple articles are tested, average the total weight differences to obtain the Moisture Retention.

Optional Components

The lathering, cleansing composition of the present invention may contain a variety of other components such as are conventionally used in a given product type provided that they do not unacceptably alter the benefits of the invention. These optional components should be suitable for application to human skin and hair, that is, when incorporated into the article they are suitable for use in contact with human skin without undue toxicity, incompatibility, instability, allergic response, and the like, within the scope of sound medical or formulator's judgment.

Preferred compositions optionally contain a safe and effective amount of therapeutic benefit agent selected from the group consisting of vitamin compounds, skin treating agents, anti-acne actives, anti-wrinkle actives, anti-skin atrophy actives, anti-inflammatory actives, topical anesthetics, artificial tanning actives and accelerators, anti-microbial actives, anti-fungal actives, sunscreen actives, anti-oxidants, skin exfoliating agents, and combinations thereof. As used herein, “a safe and effective amount” means an amount of a compound or component sufficient to significantly induce a positive effect or benefit, but low enough to avoid serious side effects, (e.g., undue toxicity or allergic reaction), i.e., to provide a reasonable benefit to risk ratio, within the scope of sound medical judgment.

The optional components useful herein can be categorized by their therapeutic or aesthetic benefit or their postulated mode of action. It is to be understood, however, that the optional components useful herein can in some instances provide more than one therapeutic or aesthetic benefit or operate via more than one mode of action. Therefore, classifications herein are made for the sake of convenience and are not intended to limit the component to that particular application or applications listed. Also, when applicable, the pharmaceutically-acceptable salts of the components are useful herein.

Co-Surfactants

The cleansing compositions of the present invention may optionally contain one or more co-surfactants. Ideally, the co-sufactants will contribute to the cleansing and lathering capabilities of the resultant composition but will not, however, contribute to the hotmelt character of the compositions. Therefore, suitable co-surfactants would those that are not deemed “mild crystalline surfactants” as described herein. Preferably, these co-surfactants will also be lathering surfactants and may be selected from the group consisting of anionic lathering surfactants, cationic lathering surfactants, nonionic lathering surfactants, amphoteric lathering surfactants, and combinations thereof.

Anionic Lathering Surfactants

Nonlimiting examples of anionic lathering surfactants useful in the compositions of the present invention are disclosed in U.S. Pat. No. 3,929,678, to Laughlin et al., issued Dec. 30, 1975.

A wide variety of anionic surfactants are potentially useful herein. Nonlimiting examples of anionic lathering surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, and combinations thereof.

Nonionic Lathering Surfactants

Nonlimiting examples of nonionic lathering surfactants for use in the compositions of the present invention include those selected from the group consisting of alkyl glucosides, alkyl polyglucosides, polyhydroxy fatty acid amides, alkoxylated fatty acid esters, sucrose esters, amine oxides, and mixtures thereof.

Cationic Lathering Surfactants

Cationic lathering surfactants are also useful in the articles of the present invention. Suitable cationic lathering surfactants include, but are not limited to, fatty amines, di-fatty quaternary amines, tri-fatty quaternary amines, imidazolinium quaternary amines, and combinations thereof. Suitable fatty amines include monalkyl quaternary amines such as cetyltrimethylammonium bromide. A suitable quaternary amine is dialklamidoethyl hydroxyethylmonium methosulfate. The fatty amines, however, are preferred. It is preferred that a lather booster is used when the cationic lathering surfactant is the primary lathering surfactant of the cleansing component. Additionally, nonionic surfactants have been found to be particularly useful in combination with such cationic lathering surfactants.

Amphoteric Lathering Surfactants

The term “amphoteric lathering surfactant,” as used herein, is also intended to encompass zwitterionic surfactants, which are well known to formulators skilled in the art as a subset of amphoteric surfactants.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof.

Vitamin Compounds

The present composition may comprise vitamin compounds, precursors, and derivatives thereof. These vitamin compounds may be in either natural or synthetic form. Suitable vitamin compounds include, but are not limited to, Vitamin A (e.g., beta carotene, retinoic acid, retinol, retinoids, retinyl palmitate, retinyl proprionate, etc.), Vitamin B (e.g., niacin, niacinamide, riboflavin, pantothenic acid, etc.), Vitamin C (e.g., ascorbic acid, etc.), Vitamin D (e.g., ergosterol, ergocalciferol, cholecalciferol, etc.), Vitamin E (e.g., tocopherol acetate, etc.), and Vitamin K (e.g., phytonadione, menadione, phthiocol, etc.) compounds.

In particular, the composition of the present invention may comprise a safe and effective amount of a vitamin B₃ compound. Vitamin B₃ compounds are particularly useful for regulating skin condition as described in U.S. Pat. No. 6,238,678 issued to Oblong on May 29, 2001. The compositions of the present invention preferably comprise from about 0.01% to about 50%, more preferably from about 0.1% to about 10%, even more preferably from about 0.5% to about 10%, and still more preferably from about 1% to about 5%, most preferably from about 2% to about 5%, of the vitamin B₃ compound.

As used herein, “vitamin B₃ compound” means a compound having the formula:

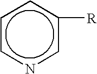

wherein R is —CONH₂ (i.e., niacinamide), —COOH (i.e., nicotinic acid) or —CH₂OH (i.e., nicotinyl alcohol); derivatives thereof; and salts of any of the foregoing.

Skin Treating Agents

The composition of the present invention may contain one or more skin treating agents. Suitable skin treating agents include those effective for preventing, retarding, arresting, and/or reversing skin wrinkles. Examples of suitable skin treating agents include, but are not limited to, alpha-hydroxy acids such as lactic acid and glycolic acid and beta-hydroxy acids such as salicylic acid.

Anti-Acne Actives

Examples of useful anti-acne actives for the composition of the present invention include, but are not limited to, the keratolytics such as salicylic acid (o-hydroxybenzoic acid), derivatives of salicylic acid such as 5-octanoyl salicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids; and bile salts such as scymnol sulfate and its derivatives, deoxycholate, and cholate.

Anti-Wrinkle and Anti-Skin Atrophy Actives

Examples of anti-wrinkle and anti-skin atrophy actives useful for the composition of the present invention include, but are not limited to, retinoic acid and its derivatives (e.g., cis and trans); retinol; retinyl esters; niacinamide, salicylic acid and derivatives thereof; sulfur-containing D and L amino acids and their derivatives and salts, particularly the N-acetyl derivatives, a preferred example of which is N-acetyl-L-cysteine; thiols, e.g., ethane thiol; hydroxy acids, phytic acid, lipoic acid; lysophosphatidic acid, and skin peel agents (e.g., phenol and the like).

Non-Steroidal Anti-inflammatory Actives (NSAIDS)

Examples of NSAIDS useful for the composition of the present invention include, but are not limited to, the following categories: propionic acid derivatives; acetic acid derivatives; fenamic acid derivatives; biphenylcarboxylic acid derivatives; and oxicams. All of these NSAIDS are fully described in U.S. Pat. No. 4,985,459 to Sunshine et al., issued Jan. 15, 1991. Examples of useful NSAIDS include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Also useful are the steroidal anti-inflammatory drugs including hydrocortisone and the like.

Topical Anesthetics

Examples of topical anesthetic drugs useful for the composition of the present invention include, but are not limited to, benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Artificial Tanning Actives and Accelerators

Examples of artificial tanning actives and accelerators useful for the composition of the present invention include, but are not limited to, dihydroxyacetaone, tyrosine, tyrosine esters such as ethyl tyrosinate, and phospho-DOPA.

Antimicrobial and Antifungal Actives

Examples of antimicrobial and antifungal actives useful for the composition of the present invention include, but are not limited to, β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole.

Anti-Viral Agents

The composition of the present invention may further comprise one or more anti-viral agents. Suitable anti-viral agents include, but are not limited to, metal salts (e.g., silver nitrate, copper sulfate, iron chloride, etc.) and organic acids (e.g., malic acid, salicylic acid, succinic acid, benzoic acid, etc.). In particular compositions which contain additional suitable anti-viral agents include those described in U.S. Pat. No. 6,294,186 issued to Beerse et al on Sep. 25, 2001, copending U.S. patent applications Ser. Nos. 09/421,131 (Biedermann et al.) and 09/421,179 (Page et al.), both of which were filed on Oct. 19, 1999.

Enzymes

The composition of the present invention may optionally include one or more enzymes. Preferably, such enzymes are dermatologically acceptable. Suitable enzymes include, but are not limited to, keratinase, protease, amylase, subtilisin, etc.

Sunscreen Actives

Also useful herein are sunscreening actives. A wide variety of sunscreening agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology. Nonlimiting examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991. Especially preferred examples of these sunscreens include those selected from the group consisting of 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof. Exact amounts of sunscreens which can be employed will vary depending upon the sunscreen chosen and the desired Sun Protection Factor (SPF) to be achieved.

Hydrocolloids

Hydrocolloids may also be optionally included in the composition of the present invention. Hydrocolloids are well known in the art and are helpful in extending the useful life of the surfactants contained in the cleansing component of the present invention such that the articles may last throughout at least one entire showering or bathing experience. Suitable hydrocolloids include, but are not limited to, xanthan gum, carboxymethyl cellulose, hydroxyethyl cellulose, hydroxylpropyl cellulose, methyl and ethyl cellulose, natural gums, gudras guar gum, bean gum, natural starches, deionitized starches (e.g., starch octenyl succinate) and the like.

Exothermic Zeolites

Zeolites and other compounds which react exothermically when combined with water may also be optionally included in the composition of the present invention.

Hydrogel Forming Polymeric Gelling Agents

In certain embodiments of the present invention, the compositions may optionally comprise an aqueous gel, i.e., a “hydrogel”, formed from a hydrogel forming polymeric gelling agent and water. More specifically, the hydrogel is contained within the cleansing component or the therapeutic benefit component of the article. When an aqueous gel is present, the articles preferably comprise from about 0.1% to about 100%, by weight of the water insoluble substrate, more preferably from about 3% to about 50%, and most preferably from about 5% to about 35%, of a hydrogel forming polymeric gelling agent, calculated based on the dry weight of the hydrogel forming polymeric gelling agent.

Suitable hydrogel forming polymeric gelling agents in the form of particles are commercially available from Hoechst Celanese Corporation, Portsmouth, Va., USA (Sanwet™ Superabsorbent Polymers) Nippon Shokubai, Japan (Aqualic™, e.g., L-75, L-76) and Dow Chemical Company, Midland, Mich., USA (Dry Tech™).

Hydrogel forming polymeric gelling agents in the form of fibers are commercially available from Camelot Technologies Inc., Leominster, Mass., USA (Fibersorb™, e.g., SA 7200H, SA 7200M, SA 7000L, SA 7000, and SA 7300).

Chelators

The hotmelt composition of the present invention may also comprise a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation that can contribute to excessive scaling or skin texture changes and against other environmental agents, which can cause skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; U.S. Pat. No. 5,462,963, issued Nov. 31, 1995 to Bush et al., U.S. Pat. No. 5,364,617, issued Nov. 15, 1994 to Bush et al. Preferred chelators useful in compositions of the subject invention are furildioxime and derivatives thereof.

Flavonoids

The composition of the present invention may optionally comprise a flavonoid compound. Flavonoids are broadly disclosed in U.S. Pat. Nos. 5,686,082, 5,686,367, and 6,093,411.

Favonoid compounds are preferably present in the instant invention at concentrations of from about 0.01% to about 20%, more preferably from about 0.1% to about 10%, and most preferably from about 0.5% to about 5%.

Sterols

The composition of the present invention may comprise a safe and effective amount of one or more sterol compounds. Examples of useful sterol compounds include sitosterol, stigmasterol, campesterol, brassicasterol, lanosterol, 7-dehydrocholesterol, and mixtures thereof. These can be synthetic in origin or from natural sources, e.g., blends extracted from plant sources (e.g., phytosterols).

Anti-Cellulite Agents

The composition of the present invention may also comprise a safe and effective amount of an anti-cellulite agent. Suitable agents may include, but are not limited to, xanthine compounds (e.g., caffeine, theophylline, theobromine, and aminophylline).

Skin Lightening Agents

The composition of the present invention may comprise a skin lightening agent. When used, the compositions preferably comprise from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof, e.g., magnesium ascorbyl phosphate or sodium ascorbyl phosphate or other salts of ascorbyl phosphate. Skin lightening agents suitable for use herein also include those described in U.S. Ser. No. 08/479,935, filed on Jun. 7,1995 in the name of Hillebrand; and U.S. Pat. No. 6,068,836, issued May 30, 2000 to Kvalnes et al.

Fillers/Binders

The compositions of the present invention may optionally include fillers and/or binders. Fillers do not have a melt point in the relevant temperature range but can be used in conjunction with a temperature sensitive surfactant phase. Fillers are well known in the art of bar soap making and may include but are not limited to: Nylons (e.g., Orgasol 1002® from Elf Atochem), Talc, Kaolin, Bentonite Clay (e.g., Bentone E W from Rheox), and cellulose.

Layer Binders

The composition of the present invention may optionally comprise binders. Binders or binding materials are useful for sealing the various layers of the present articles to one another thereby maintaining the integrity of the article. The binders may be in a variety of forms including, but not limited to, spray on, webs, separate layers, binding fibers, etc. Suitable binders may comprise latexes, polyamides, polyesters, polyolefins and combinations thereof.

Hydrophobic Conditioning Agents

The articles of the present invention may comprise one or more hydrophobic conditioning agents which are useful for providing a conditioning benefit to the skin :or hair during the use of the article. The articles of present invention preferably comprise from about 0.5% to about 1,000%, more preferably from about 1% to about 200%, and most preferably from about 10% to about 100%, by weight of the water insoluble substrate, of a hydrophobic conditioning agent.

The hydrophobic conditioning agent may be selected from one or more hydrophobic conditioning agents such that the weighted arithmetic mean solubility parameter of the hydrophobic conditioning agent is less than or equal to 10.5.

Nonlimiting examples of hydrophobic conditioning agents include those selected from the group consisting of mineral oil, petrolatum, lecithin, hydrogenated lecithin, lanolin, lanolin derivatives, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C4-C20 alkyl ethers, di C8-C30 alkyl ethers, and combinations thereof.

Straight and branched chain hydrocarbons having from about 7 to about 40 carbon atoms are useful herein. Nonlimiting examples of these hydrocarbon materials include dodecane, isododecane, squalane, cholesterol, hydrogenated polyisobutylene, docosane (i.e. a C₂₂ hydrocarbon), hexadecane, isohexadecane (a commercially available hydrocarbon sold as Permethyl® 101A by Presperse, South Plainfield, N.J.). Also useful are the C7-C40 isoparaffins, which are C7-C40 branched hydrocarbons. Polydecene, a branched liquid hydrocarbon, is also useful herein and is commercially available under the trade names Puresyn 100® and Puresyn 3000® from Mobile Chemical (Edison, N.J.).

Also useful are various C1-C30 monoesters and polyesters of sugars and related materials. Suitable ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985.

Nonvolatile silicones such as polydialkylsiloxanes, polydiarylsiloxanes, and polyalkarylsiloxanes are also useful oils. These silicones are disclosed in U.S. Pat. No. 5,069,897, to Orr, issued Dec. 3, 1991. The polyalkylsiloxanes correspond to the general chemical formula R₃SiO[R₂SiO]ₓSiR₃ wherein R is an alkyl group (preferably R is methyl or ethyl, more preferably methyl) and x is an integer up to about 500, chosen to achieve the desired molecular weight. Commercially available polyalkylsiloxanes include the polydimethylsiloxanes, which are also known as dimethicones, nonlimiting examples of which include the Vicasil® series sold by General Electric Company and the Dow Corning® 200 series sold by Dow Corning Corporation. Specific examples of polydimethylsiloxanes useful herein include Dow Corning® 225 fluid having a viscosity of 10 centistokes and a boiling point greater than 200° C., and Dow Corning® 200 fluids having viscosities of 50, 350, and 12,500 centistokes, respectively, and boiling points greater than 200° C. Also useful are materials such as trimethylsiloxysilicate, which is a polymeric material corresponding to the general chemical formula [(CH₂)₃SiO_{1/2}]ₓ[SiO₂]y, wherein x is an integer from about 1 to about 500 and y is an integer from about 1 to about 500. A commercially available trimethylsiloxysilicate is sold as a mixture with dimethicone as Dow Corning® 593 fluid. Also useful herein are dimethiconols, which are hydroxy terminated dimethyl silicones. These materials can be represented by the general chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer up to about 500, chosen to achieve the desired molecular weight. Commercially available dimethiconols are typically sold as mixtures with dimethicone or cyclomethicone (e.g. Dow Corning® 1401, 1402, and 1403 fluids). Also useful herein are polyalkylaryl siloxanes, with polymethylphenyl siloxanes having viscosities from about 15 to about 65 centistokes at 25° C. being preferred. These materials are available, for example, as SF 1075 methylphenyl fluid (sold by General Electric Company) and 556 Cosmetic Grade phenyl trimethicone fluid (sold by Dow Corning Corporation). Alkylated silicones such as methyldecyl silicone and methyloctyl silicone are useful herein and are commercially available from General Electric Company. Also useful herein are alkyl modified siloxanes such as alkyl methicones and alkyl dimethicones wherein the alkyl chain contains 10 to 50 carbons. Such siloxanes are commercially available under the tradenames ABIL WAX 9810® (C₂₄-C₂₈ alkyl methicone) (sold by Goldschmidt) and SF1632 (cetearyl methicone)(sold by General Electric Company).

Vegetable oils and hydrogenated vegetable oils are also useful herein.

Also useful are C4-C20 alkyl ethers of polypropylene glycols, C1-C20 carboxylic acid esters of polypropylene glycols, and di-C8-C30 alkyl ethers. Nonlimiting examples of these materials include PPG-14 butyl ether, PPG-15 stearyl ether, dioctyl ether, dodecyl octyl ether, and mixtures thereof.

Hydrophilic Conditioning Agents

The compositions and articles of the present invention may optionally comprise one or more hydrophilic conditioning agents as therapeutic benefit agents. Nonlimiting examples of hydrophilic conditioning agents include those selected from the group consisting of polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof. Specific examples of useful hydrophilic conditioning agents include materials such as urea; guanidine; glycolic acid and glycolate salts (e.g., ammonium and quaternary alkyl ammonium); lactic acid and lactate salts (e.g., ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-30, PEG-50, polypropylene glycols such as PPG-9, PPG-12, PPG-15, PPG-17, PPG-20, PPG-26, PPG-30, PPG-34; alkoxylated glucose; hyaluronic acid; cationic skin conditioning polymers (e.g., quaternary ammonium polymers such as Polyquaternium polymers); and mixtures thereof. Glycerol, in particular, is a preferred hydrophilic conditioning agent in the articles of the present invention. Also useful are materials such as aloe vera in any of its variety of forms (e.g., aloe vera gel), chitosan and chitosan derivatives, e.g., chitosan lactate, lactamide monoethanolamine; acetamide monoethanolamine; and mixtures thereof. Also useful are propoxylated glycerols as described in propoxylated glycerols described in U.S. Pat. No. 4,976,953, to Orr et al., issued Dec. 11, 1990.

Preferred cleansing compositions that include therapeutic benefit agents are in the form of an emulsion, which further comprises an aqueous phase and an oil phase. As will be understood by the skilled artisan, a given component will distribute primarily into either the aqueous or oil phase, depending on the water solubility/dispersibility of the therapeutic benefit agent in the component. In one embodiment, the oil phase comprises one or more hydrophobic conditioning agents. In another embodiment, the aqueous phase comprises one or more hydrophilic conditioning agents.

Structured Conditioning Agents

The therapeutic benefit agents of the present invention may be structured conditioning agents. Suitable structured conditioning agents include, but are not limited to, vesicular structures such as ceramides, liposomes, and the like.

In another embodiment, the therapeutic benefit agents are comprised within a coacervate-forming composition. Preferably, the coacervate-forming composition comprises a cationic polymer, an anionic surfactant, and a dermatologically acceptable carrier for the polymer and surfactant.

Methods of Cleansing and Conditioning Skin or Hair

The present invention also relates to a method of cleansing the skin or hair with the composition of the present invention. These methods comprise the steps of: a) wetting with water a lathering, cleansing composition comprising: a safe and effective amount of a mild crystalline surfactant, water, and a safe and effective amount of an anhydrous solvent, wherein the composition exhibits hotmelt behavior and is substantially free of additional water-soluble crystalline structurants besides the crystalline surfactants and b) contacting the skin or hair with the wetted composition. In additional embodiments, such methods may also comprise the use of a substantially dry, water insoluble substrate in combination with the above-described lathering, cleansing composition such that a substantially dry cleansing and conditioning substrate article is thereby formed.

The compositions of the present invention are water-activated and are therefore intended to be wetted with water prior to use. As used herein, “water-activated” means that the present invention is presented to the consumer in semi-solid or solid form to be used after wetting with water. It is found that when the compositions of the present invention include a lathering surfactant as the mild crystalline surfactant the compositions produce lather or are “activated” upon contact with water and further agitation. Accordingly, the composition is wetted by immersion of it or a substrate article containing the composition in water or by placing either under a stream of water. When the composition of the present invention comprises a mild crystalline surfactant that is a lathering surfactant, lather may be generated from the composition and any substrate article containing the composition by mechanically agitating and/or deforming the article either prior to or during contact of the article with the skin or hair. The resulting lather is useful for cleansing the skin or hair. During the cleansing process and subsequent rinsing with water, any therapeutic or aesthetic benefit agents are deposited onto the skin or hair. Deposition of the therapeutic or aesthetic benefit agents is enhanced by the physical contact of the substrate with the skin or hair as well by the inclusion of one or more deposition aids.

### EXAMPLES

The following examples further describe and demonstrate embodiments within the scope of the present invention. In the following examples, all ingredients are listed at an active level. The examples are given solely for the purpose of illustration and are not to be construed as limitations of the present invention, as many variations thereof are possible without departing from the spirit and scope of the invention. Where not specifically mentioned, the ingredients are mixed in a manner consistent with that method of mixing practiced by one skilled in the art.

Ingredients are identified by chemical or CTFA name.

Manufacturing Method for Present Compositions

The following directions for preparing the compositions of present invention may be followed generally in making each of the exemplary formulations.

Premix the polymers (e.g. Polyquat-10, PEG-90M), citric acid & propylene glycol in an agitated vessel until well dispersed. Add the co-surfactant (e.g., lauramidopropyl betaine), preservatives, chelants & dye. Mix thoroughly until powders are hydrated. Add the mild crystalline surfactant (e.g., sodium cocoglyceryl ether sulfonate) to the premix & heat to 60° C. until it melts.

In the case where a “two-in-one” composition is needed, add the lipids & waxes pre-melted at 60° C. in an oven.

Mix the composition with high shear (e.g. rotor stator mixer) & agitation until homogeneous (in 2-in-1 compositions the amount & time of high shear will pre-determine the particle size of the emulsified waxes & lipids).

Cool to 55° C., add the perfume oil & mix for a further 10 minutes.

Pour the hot mix into a suitable container to solidify, the compositions can be remelted as required at 55-60° C. for use as hotmelts.

### Example 1

A lathering, cleansing composition is prepared as described above. May be combined with a therapeutic benefit agent or paired with a therapeutic benefit component that for use.

| | | % |
|---|---|---|
| Ingredient | Wt % | Water |
| Sodium Cocoglyceryl Ether Sulfonate | 62.91 | 26.2 |
| Lauramidopropyl Betaine* | 19.67 | 12.8 |
| Methylchloroisothiazolinone & | 0.03 | |
| Methylisothiazolinone | | |
| Disodium EDTA | 0.10 | |
| Sodium Benzoate | 0.25 | |
| Polyquaternium-10 | 0.50 | |
| PEG 90M | 0.20 | |
| Citric Acid Anhydrous | 0.13 | |
| Propylene Glycol | 15.21 | |
| Perfume | 1.00 | |
| FD&C Blue #1 | 0.001 | |
| Total | 100.00 | 39.0 |

*Other suitable Amphoteric surfactants are, Cocamidopropyl Hydroxy Sultaine, Sodium Lauroamphoacetate.

### Example 2

A two-in-one cleansing and conditioning composition is prepared.

| | | % |
|---|---|---|
| Ingredient | Wt % | Water |
| Sodium Cocoglyceryl Ether Sulfonate | 58.66 | 24.4 |
| Disodium Cocoamphodiproprionate | 18.14 | 11.8 |
| Methylchloroisothiazolinone & | 0.03 | |
| Methylisothiazolinone | | |
| Disodium EDTA | 0.10 | |
| Sodium Benzoate | 0.25 | |
| Polyquat-10 | 0.50 | |
| PEG 90M | 0.20 | |
| Tocopheryl Acetate | 0.20 | |
| Petrolatum* | 4.80 | |
| Lanolin Wax* | 5.00 | |
| Citric Acid Anhydrous | 0.15 | |
| Propylene Glycol | 11.09 | |
| Perfume | 1.00 | |
| Red 40 | 0.001 | |
| Total | 100.00 | 36.2 |

Other suitable Lipids & Waxes include Mycrocrystalline wax. Cetearyl Methicone. C30-C45 Alkyl Methicone. Tribehenin. Hydrogenated Sunflower wax & other waxes having a melt point 30-70° C.

### Example 3

A lathering, cleansing composition is prepared as described above.

| Ingredient | Wt % | % Water |
|---|---|---|
| Sodium Cocoglyceryl Ether Sulfonate | 60.00 | 25.0 |
| Disodium Cocoamphodiproprionate | 16.25 | 9.8 |
| Methylchloroisothiazolinone & | 0.03 | |
| Methylisothiazolinone | | |
| Disodium EDTA | 0.10 | |
| Sodium Benzoate | 0.25 | |
| Guar Hydroxypropyl Trimonium | 0.50 | |
| Chloride | | |
| PEG 90M | 0.20 | |
| Tocopheryl Acetate | 0.20 | |
| Petrolatum | 4.80 | |
| Cetyl Alcohol | 5.00 | |
| Citric Acid Anhydrous | 0.15 | |
| Propylene Glycol | 11.52 | |
| Perfume | 1.00 | |
| Red 40 | 0.001 | |
| Total | 100.00 | 34.8 |

### Example 4

A two-in-one cleansing and conditioning composition is prepared.

| | | % |
|---|---|---|
| Ingredient | Wt % | Water |
| Sodium Cocoglyceryl Ether Sulfonate | 54.86 | 22.5 |
| Lauramidopropyl Betaine | 16.98 | 11.0 |
| Methylchloroisothiazolinone & | 0.03 | |
| Methylisothiazolinone | | |
| Disodium EDTA | 0.10 | |
| Sodium Benzoate | 0.25 | |
| Polyquaternium-10 | 0.50 | |
| PEG 90M | 0.50 | |
| Tocopheryl Acetate | 0.20 | |
| Petrolatum | 7.30 | |
| Lanolin Wax | 7.50 | |
| Citric Acid Anhydrous | 0.15 | |
| Propylene Glycol | 10.65 | |
| Perfume | 1.00 | |
| Red 40 | 0.001 | |
| Total | 100.00 | 33.5 |

### Example 5

A two-in-one cleansing and conditioning composition is prepared.

| | | % |
|---|---|---|
| Ingredient | Wt % | Water |
| Sodium Cocoglyceryl Ether Sulfonate | 60.00 | 25.0 |
| Lauramidopropyl Betaine | 18.57 | 12.0 |
| Methylchloroisothiazolinone & | 0.03 | |
| Methylisothiazolinone | | |
| Disodium EDTA | 0.10 | |
| Sodium Benzoate | 0.25 | |
| Guar Hydroxypropyl Trimonium | 0.50 | |
| Chloride | | |
| PEG 90M | 0.20 | |
| Tocopheryl Acetate | 0.20 | |
| Petrolatum | 2.3 | |
| Lanolin Wax | 2.5 | |
| Citric Acid Anhydrous | 0.15 | |
| Propylene Glycol | 14.20 | |
| Perfume | 1.00 | |
| Red 40 | 0.001 | |
| Total | 100.00 | 37 |

### Example 6

A two-in-one cleansing and conditioning composition is prepared to provide intense moisturizing.

| | | % |
|---|---|---|
| Ingredient | Wt % | Water |
| Sodium Cocoglyceryl Ether Sulfonate | 58.65 | 24.4 |
| Lauramidopropyl Betaine | 18.15 | 11.8 |
| Methylchloroisothiazolinone & | 0.03 | |
| Methylisothiazolinone | | |
| Disodium EDTA | 0.10 | |
| Sodium Benzoate | 0.25 | |
| Polyquaternium-10 | 0.50 | |
| PEG 14M | 0.50 | |
| Tocopheryl Acetate | 1.00 | |
| Shea Butter (Butyrospermum Parkii)* | 9.67 | |
| Citric Acid Anhydrous | 0.15 | |
| Propylene Glycol | 10.00 | |
| Perfume | 1.00 | |
| Red 40 | 0.001 | |
| Total | 100.00 | 36.2 |

*Other suitable fats & butters are Illipe Fat, Sal Fat, Palm Oil Stearine Fraction.

### Example 7A-7G

Compositions of examples 1-6 are melted at 60° C., when liquid the composition is coated (4.0-6.0 grams per application) onto a substrate and allowed to cool & solidify.

Additionally a therapeutic benefit component (as disclosed below) is melted at 50° C., the composition is coated (0.5-2.0 grams per application) onto the same substrate adjacent to the surfactant composition.

Method of coating the compositions may include, but are not limited to, sprinkling, simple extrusion, dip coating, spraying, slot coating, roll transfer (e.g., pressure roll or kiss roll), gravure printing, and a dipping & nipping application, no additional drying is necessary.

Therapeutic benefit component example:

| Ingredient | Wt % |
|---|---|
| Petrolatum | 24.00 |
| Lanolin wax | 59.95 |
| Cetearyl Methicone | 10.00 |
| Microcrystalline wax | 5.00 |
| Tocopheryl Acetate | 1.00 |
| Red 7 pigment in castor oil | 0.05 |
| Total | 100.00 |

Melt all ingredients at 80° C. in a heated vessel, mix until pigment is well dispersed and allow to cool in suitable container until required for use.

When more than on substrate layer is employed for the articles of the present invention then a sheet of the second layer is then placed on the sheet of the first layer, preferably, but not always, over the cleansing composition and/or therapeutic benefit component. The sheets are sealed together by a conventional sealing method that may include, but is not limited to, heat, pressure, glue, ultrasound, etc.

Suitable substrate layers compositions:

| Substrates | Basis wt | Manufacturer |
|---|---|---|
| High loft blend of PET heat bonded with | 84 gsm | Libeltex N.V. |
| 70% PET/PE bicomponent fiber. | | |
| Hydroentangled 75% PP 25% Rayon | 75 gsm | Suominen |
| blend. | | |
| Spunbond PE/PP bicomponent fiber | 30 gsm | BBA |
| | | Nonwovens |
| Apertured Hydroentangled 30% rayon/ | 78 gsm | PGI Nonwovens |
| 70% Polyester blend | | |

The coated laminated article may be cut into any suitable shape or size that is most appropriate for its intended use (e.g. in the shower).

### Example 8

Lathering, Cleansing Compositions in the Form of Capsules are Prepared.

Compositions of examples 1-6 are melted at 60° C., when liquid the compositions are poured into cooled moulds to set into a ‘capsule’ like form. For quick release from the moulds and to prevent stickiness in use, the capsules may be coated with a powder (e.g. Nylon-12) or a waxy layer (e.g. stearyl alcohol). Such crystallised capsules may be easily used in the bath or at a sink as they slowly dissolve in warm water thereby giving a sustained release of cleansing & therapeutic agents.

Optionally the capsules can be inserted into a body sponge or other bath implement for high foaming applications.

Additional Examples

The specification of co-pending U.S. Ser. No. 09/785,882 filed on Feb. 16, 2001 in the names of Lorenzi et al. is incorporated herein by reference. Accordingly, Applicants further illustrate that the compositions of the present invention may be used in place of the compositions disclosed in Examples 140-154 of this co-pending application to yield the presently claimed articles.

What is claimed is:

1. A lathering cleansing composition comprising:
a. a safe and effective amount of one or more mild crystalline surfactants,
b. water; and
c. a safe and effective amount of one or more polar solvents other than water
wherein the composition exhibits hotmelt behavior and is substantially free of additional water soluble crystalline structurants.

2. The composition of claim 1 wherein the composition further comprises one or more therapeutic benefit agents.

3. The composition of claim 2 wherein said therapeutic benefit agent is selected from the group consisting of vitamin compounds, skin treating agents, anti-acne actives, anti-wrinkle actives, anti-skin atrophy actives, anti-inflammatory actives, topical anesthetics, artificial tanning actives and accelerators, anti-microbial actives, anti-fungal actives, anti-viral agents, enzymes, sunscreen actives, anti-oxidants, skin exfoliating agents, and combinations thereof.

4. The composition of claim 1 wherein said mild crystalline surfactant is selected from the group consisting of alkyl sulfonates, acyl isethionates, alkyl glucosides, glucose amides, alkyl lactylates, dialkyl sulfosuccinates, and combinations thereof.

5. The composition of claim 1 wherein said composition comprises at least about 25%, by weight of the composition, of water.

6. The composition of claim 1 wherein said composition comprises from about 15% to about 45%, by weight of the composition, of the mild crystalline surfactant.

7. The composition of claim 1 wherein said composition comprises from about 1% to about 25%, by weight of the composition, of the polar solvent.

8. The composition of claim 1 wherein said composition is formed into a product selected from the group consisting of capsules, tablets, and combinations thereof.

9. A substantially dry, disposable personal care article suitable for cleansing and conditioning skin and hair, said article comprising:
a. a water insoluble substrate comprising a nonwoven layer and
b. the composition of claim 1 contacted with said substrate.

10. The article of claim 9 wherein said composition further comprises a therapeutic benefit agent.

11. The article of claim 9 that additionally comprises a therapeutic benefit component that further comprises a therapeutic benefit agent wherein said benefit component and said composition are contacted with the substrate in areas distinct from one another.
